(19) United States
(12) Patent Application Publication
(10) Pub. No.: US 2002/0133099 A1
(43) Pub. Date: Sep. 19, 2002

(54) Multiple source shockwave device

(76) Inventor: Moshe Ein-Gal, Ramat Hasharon (IL)

Correspondence Address: David Klein, DEKEL PATENT LTD., 12 HaEgoz Street, Apt. 4, REHOVOT, IL

(21) Appl. No.: 09/804,196

(22) Filed: Mar. 13, 2001

(51) Int. Cl. A61H 1/00; A61H 1/02; A61H 5/00; A61B 17/20; A61B 8/00; A61B 8/12; A61B 8/14

(52) U.S. Cl. 601/2; 600/439; 604/22

(57) ABSTRACT

A shockwave device including a plurality of shockwave sources, at least one of the sources having a shockwave propagation axis, the sources being adapted to deliver shockwave energy to a patient.

### FIELD OF THE INVENTION

The present invention relates to extracorporeal shockwave treatment (ESWT) in general, and particularly to a multiple shockwave source device.

### BACKGROUND OF THE INVENTION

Extracorporeal shockwave treatment (ESWT) is an extra-corporeal treatment modality for a variety of applications including disintegration of urinary tract calculi, disintegration of any stone-like concretions or depositions of minerals and salts found in ducts, blood vessels or hollow organs of a patient's body, advancing bone union by causing micro-fractures and relieving pain associated with tendons, joints and bony structures. A shockwave device is a device used to perform ESWT, which includes a shockwave source typically comprising an electrical-to-shockwave energy converter and a focusing mechanism for directing shockwaves energy to treated area. Electro-hydraulic, electromagnetic and piezo-electric are some of the technologies utilized for energy conversion while focusing is accomplished via acoustic lenses or via ellipsoidal, parabolic or other shaped reflector. Typically, a shockwave focusing mechanism is cylindrically symmetric about an axis defining the shockwave propagation axis.

One well-known example of ESWT is extracorporeal shockwave lithotripsy (ESWL), which is an extra-corporeal treatment modality for disintegration of calculi, such as kidney stones, stone-like concretions in ducts or hollow organs, and other brittle deposits in the body. A lithotripter is a device used to perform ESWL, which includes a shockwave head, typically comprising a semi-ellipsoidal reflector, to deliver shockwave energy to disintegrate the calculi.

In an electro-hydraulic shockwave head, capacitor-stored energy is electrically discharged underwater between closely spaced electrodes in the semi-ellipsoidal reflector. An ellipsoid of revolution has two focal points. The semi-ellipsoidal reflector comprises a truncated ellipsoid which has a pair of electrodes spaced apart to define a spark gap substantially at the first focus point of the ellipsoid. A rubber or elastomeric diaphragm covers the truncated, open end of the semi-ellipsoidal reflector. The reflector is filled with water having a sufficient saline content to make it conductive. The reflector is positioned with the diaphragm at the end thereof against the patient's body such that the second focus point of the reflector lies substantially on the calculus to be disintegrated. High voltage electrical pulses generate a series of sparks in the gap between the electrodes. Each such spark flashes a certain amount of water into steam, and may actually dissociate a certain amount of the water. A shockwave is generated which is reflected by the semi-ellipsoidal reflector to focus substantially on the calculus. The shockwave energy passes through the water in the reflector, through the diaphragm, and through human tissue, which is mostly water. Within an hour, the calculus is usually reduced to fine particles. In the case of kidney stones, the fine particles pass from the body along with urine.

Other shockwave sources are also used in the prior art for generating shock waves. An electromagnetic shockwave head uses a short high-current pulse to drive a speaker-like membrane in order to initiate the wave. A piezo-electric shockwave head works on basically the same principle as the electromagnetic shockwave head.

Target localization and disintegration assessment in lithotripsy are often obtained by x-ray fluoroscopy. The fluoroscope's isocenter coincides with the shockwave focus by means of mechanical coupling. The target position relative to the isocenter is obtained by imaging the target using at least two orientations, one of which is preferably vertical. The target is brought to the isocenter by moving a couch on which the patient lies. In-place fluoroscopy requires that the shockwave head does not block the fluoroscope's field-of-view.

A lithotripter couch or table, in addition to offering 3-D motion and x-ray transparency, allows contact of the patient with the shockwave head through a cutout or hole in the tabletop. The lithotripter reflector is positioned beneath the table, and the diaphragm or membrane over the open upper end of the lithotripter extends through the cutout or hole and into engagement with the patient's body. The need for such a cutout or hole prevents the use of a conventional couch.

Lithotripters are known which further include an x-ray system for locating the calculi that are to be disintegrated. For example, U.S. Pat. No. 4,984,565 to Rattner, et al., mentions in the background lithotripters that are provided with two x-ray systems, each having an x-ray source and an x-ray detector. The patient is trans-irradiated with x-rays from two directions, so that it is possible to locate a calculus to be disintegrated. Two shockwave applicators are adjustably mounted so that one of the shock wave applicators can be moved laterally to the patient for treatment. However, only one shockwave applicator is used to deliver shockwave energy at a time.

Rattner et al. describes a lithotripter with an x-ray system for locating calculi. The x-radiator for the x-ray system and the shockwave head are arranged relative to each other so that a central x-ray of the x-ray system proceeds substantially centrally through the shockwave head.

U.S. Pat. No. 5,399,146 to Nowacki et al. describes an extracorporeal isocentric lithotripter, which has a common isocentric axis of rotation. The patient lies on a table that is movable to position the target inside the patient on the isocentric axis. An X-ray emitter and an image intensifier lie on a common diameter, which is rotated about the isocentric axis in order to position the x-ray apparatus and the image intensifier in at least two positions to ascertain the location of the target. A shockwave head of the lithotripter is mounted on a support rotatable about the isocentric axis to align the shockwave head with the target. The shockwave head is mounted on the rotatable support by a double pivot arrangement to bring the second focus point of the reflector into coincidence with the target for disintegration of a concretion found at the target.

There is a problem common to lithotripters of the prior art. Since the interface between the shockwave head and the patient is not ideal, some energy dissipates on the patient's skin, causing pain and discomfort. Additional pain and tissue damage are caused by the shockwave energy passing through tissue in close proximity to the target. Pain and tissue damage set a limit to shockwave head energy.

### SUMMARY OF THE INVENTION

The present invention seeks to provide an improved shockwave device that includes multiple shockwave sources. The invention uses any combination of shockwave sources, such as, but not limited to, electro-hydraulic shockwave sources, electromagnetic shockwave sources and piezo-electric shockwave sources, for example. The shockwave sources are positioned such that their respective shockwave axes coincide at a focus. Preferably the shockwave sources are focusable such that their respective foci coincide, and the shockwaves may substantially simultaneously reach the common focus. The full brunt of the shockwaves is applied only at the common focus, which lies at the target to be disintegrated.

An advantage of the simultaneous focusing of the invention is that although shockwaves from all of the sources contribute to the focal shockwave pressure at the target, only one shockwave contributes to out-of-focus pressure at any given point away from the target. In this manner, skin, tissue and body structures, which are not desired to be treated, are subjected to significantly less pressure than in the prior art, which uses a single shockwave head or source. The invention significantly alleviates the pain and discomfort felt by the patient in prior art lithotripsy. The invention also enables using a regular couch or table with no cutout, and there is no interference with imaging equipment.

There is thus provided in accordance with a preferred embodiment of the invention a shockwave device including a plurality of shockwave sources, at least one of the sources having a shockwave propagation axis, the sources being adapted to deliver shockwave energy to a patient.

In accordance with a preferred embodiment of the invention a controller is provided which is in communication with the shockwave sources, and is adapted to control the shockwave sources.

Further in accordance with a preferred embodiment of the invention the controller is adapted to selectively orient the shockwave propagation axis.

Still further in accordance with a preferred embodiment of the invention at least one of the sources is characterized by a focus and the controller is adapted to selectively position the focus.

In accordance with a preferred embodiment of the invention at least two of the shockwave sources are characterized by a shockwave propagation axis, the sources being positioned such that their respective shockwave propagation axes coincide at a common focus.

Further in accordance with a preferred embodiment of the invention at least two of the shockwave sources are characterized by a shockwave propagation axis and a focus, the sources being focusable such that their respective foci coincide at a common focus.

Still further in accordance with a preferred embodiment of the invention the controller controls the shockwave energy delivered by the shockwave sources.

Additionally in accordance with a preferred embodiment of the invention the controller controls delivery of the shockwave energy to at least one location in accordance with a timing sequence.

In accordance with one preferred embodiment of the invention, the controller controls the shockwave sources such that all shockwaves from the shockwave sources substantially simultaneously reach the common focus.

In accordance with another preferred embodiment of the invention, the controller controls the shockwave sources such that shockwaves from the shockwave sources reach the common focus at different times. The shockwave sources may operate in series or parallel.

Further in accordance with a preferred embodiment of the invention the shockwave sources are placed generally equidistant from the common focus.

Still further in accordance with a preferred embodiment of the invention the shockwave sources are arranged to lie in a plane generally perpendicular to a longitudinal axis of a patient.

Additionally in accordance with a preferred embodiment of the invention the shockwave device includes imaging apparatus and a patient couch.

In accordance with a preferred embodiment of the invention the shockwave sources are arranged to lie in a plane generally perpendicular to a longitudinal axis of the couch.

Further in accordance with a preferred embodiment of the invention the shockwave sources are arranged with respect to the couch such that a back of a patient lying on the couch is generally perpendicular to a plane in which lie the shockwave propagation axes.

Still further in accordance with a preferred embodiment of the invention the shockwave sources are pivotally mounted on a shockwave assembly.

Additionally in accordance with a preferred embodiment of the invention at least one actuator is adapted to rotate at least one of the shockwave sources about a pivot axis in the shockwave assembly.

### BRIEF DESCRIPTION OF THE DRAWINGS

The present invention will be understood and appreciated more fully from the following detailed description taken in conjunction with the drawings in which:

FIG. 1 is a simplified pictorial illustration of a shockwave device constructed and operative in accordance with a preferred embodiment of the invention;

FIG. 2 is a simplified block diagram of shockwave sources of the shockwave device of FIG. 1, wherein shockwaves from the shockwave sources substantially simultaneously reach a common focus at a target; and

FIGS. 3 and 4 are simplified pictorial and side-view illustrations of shockwave sources mounted on a shockwave assembly, constructed and operative in accordance with a preferred embodiment of the invention.

### DETAILED DESCRIPTION OF A PREFERRED EMBODIMENT

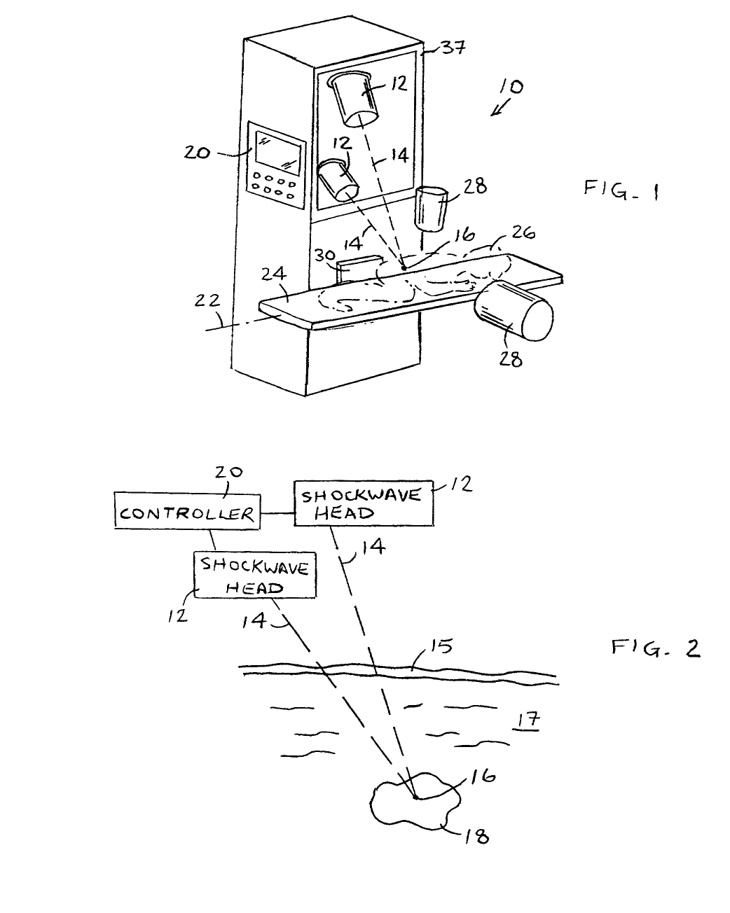

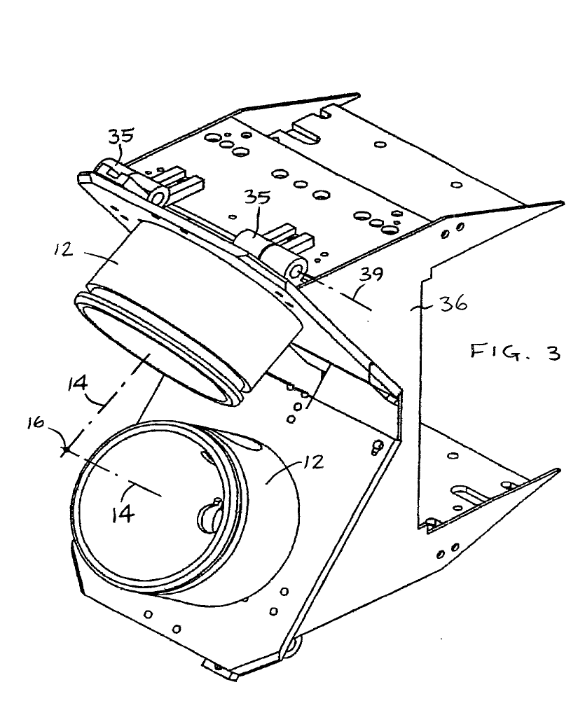

Reference is now made to FIGS. 1 and 2, which illustrate shockwave device 10, constructed and operative in accordance with a preferred embodiment of the present invention.

Shockwave device 10 preferably includes a plurality of shockwave sources 12 each characterized by a shockwave propagation axis 14. Shockwave sources 12 may be positioned such that their respective shockwave propagation axes 14 coincide at a common focus 16, located in a target 18 (FIG. 2), which is to be disintegrated. Shockwave sources 12 are preferably focusable such that their respective foci coincide at the common focus 16.

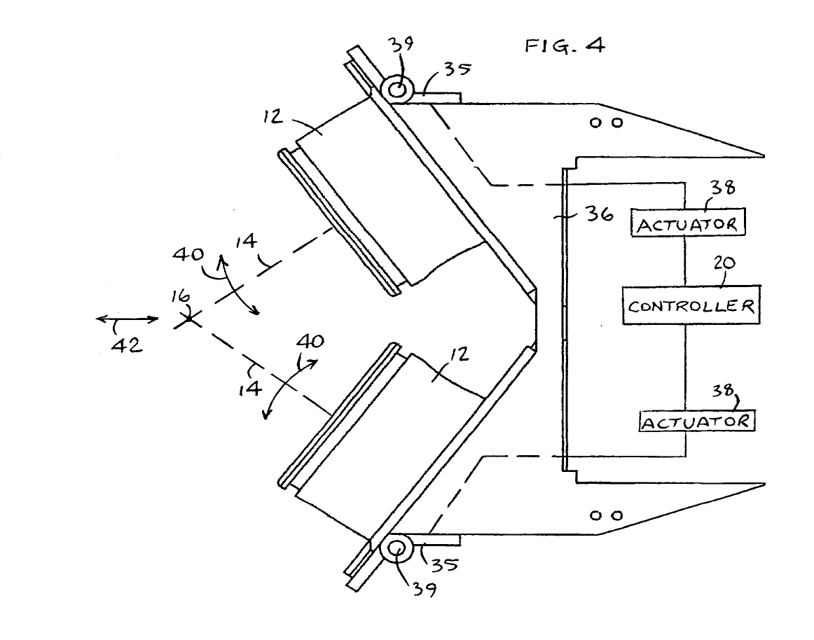

Shockwave sources 12 may include any combination of known shockwave sources, such as, but not limited to, electro-hydraulic shockwave sources, electromagnetic shockwave sources and piezo-electric shockwave sources, for example. In general, shockwave sources 12 may be mounted on a shockwave assembly 36 as shown in FIGS. 3 and 4, which may be mounted in a console 37 (FIG. 2).

A controller 20 is preferably provided, which is in wired or wireless communication with shockwave sources 12. (Controller 20 is omitted from FIG. 3 for the sake of simplicity.) Controller 20 may control the delivery of shockwaves from shockwave sources 12 to the target 18 in a variety of manners. In one embodiment of the invention, shockwave sources 12 are controlled such that all the shockwaves from the shockwave sources 12 substantially simultaneously reach the common focus 16. In another embodiment of the invention, shockwave sources 12 are controlled such that some of the shockwaves from the shockwave sources 12 reach the common focus 16 at different times. The type of shockwave delivery may be customized for any particular treatment modality. The shockwave sources 12 may operate in series or in parallel.

As seen in FIG. 2, an advantage of simultaneous focusing is that although shockwaves from all of the sources 12 contribute to the focal shockwave pressure at the target 18, only one shockwave contributes to out-of-focus pressure at any given point away from the target 18. In this manner, skin 15 or tissue 17 in the path of the shockwaves, is subjected to significantly less pressure than in the prior art, which uses a single shockwave source. The invention significantly alleviates the pain and discomfort felt by the patient in prior art lithotripsy.

The shockwave sources 12 are preferably placed generally equidistant from the common focus 16, and are preferably arranged to lie in a plane generally perpendicular to a longitudinal axis 22 of a patient couch 24 (this being generally the same longitudinal axis of a patient 26 lying on the couch 24).

Target localization and disintegration assessment are preferably obtained by imaging apparatus 28, such as x-ray fluoroscopy equipment. As seen in FIG. 1, imaging apparatus 28 may be placed at a variety of orientations with respect to patient 26. Add-on accessories 30, such as various hold down devices, backrests and the like, may be provided with couch 24, in order to maintain patient 26 on the edge of couch 24. In this manner, shockwave sources 12 may be arranged with respect to couch 24 such that the back of patient 26 is generally perpendicular to the plane in which lie the shockwave propagation axes 14. The invention thus enables using a regular couch or table with no cutout, and there is no interference with imaging apparatus 28.

As seen in FIGS. 3 and 4, shockwave sources 12 may be pivotally mounted on shockwave assembly 36, such as by means of hinged connections 35. One or more actuators 38 may be operatively connected to hinged connections 35. Actuators 38 may be controlled by controller 20 to rotate one or both of shockwave sources 12 about a pivot axis 39 of one or both of hinged connections 35. This causes the shockwave propagation axes 14 to rotate generally in the direction of arrows 40, thereby changing the position of common focus 16, as indicated by arrows 42 (FIG. 4). Thus, controller 20 is adapted to selectively orient one or more of the shockwave propagation axes 14 or to selectively position the focuses of shockwave sources 12. Furthermore, in this manner, controller 20 may control the shockwave energy delivered by shockwave sources 12, and may control delivery of the shockwave energy to one or more locations in accordance with a timing sequence.

It will be appreciated by person skilled in the art, that the present invention is not limited by what has been particularly shown and described herein above. Rather the scope of the present invention is defmed only by the claims that follow:

What is claimed is:

1. A shockwave device comprising:
a plurality of shockwave sources, at least one of said sources having a shockwave propagation axis, the sources being adapted to deliver shockwave energy to a patient.

2. The shockwave device according to claim 1 and further comprising a controller in communication with said shockwave sources, adapted to control said shockwave sources.

3. The shockwave device according to claim 2 wherein said controller is adapted to selectively orient said shockwave propagation axis.

4. The shockwave device according to claim 2 wherein at least one of said sources is characterized by a focus and said controller is adapted to selectively position said focus.

5. The shockwave device according to claim 1 wherein at least two of said shockwave sources are characterized by a shockwave propagation axis, the sources being positioned such that their respective shockwave propagation axes coincide at a common focus.

6. The shockwave device according to claim 1 wherein at least two of said shockwave sources are characterized by a shockwave propagation axis and a focus, the sources being focusable such that their respective foci coincide at a common focus.

7. The shockwave device according to claim 2 wherein said controller controls the shockwave energy delivered by said shockwave sources.

8. The shockwave device according to claim 2 wherein said controller controls delivery of the shockwave energy to at least one location in accordance with a timing sequence.

9. The shockwave device according to claim 8 wherein said controller controls said shockwave sources such that all shockwaves from said shockwave sources substantially simultaneously reach the common focus.

10. The shockwave device according to claim 8 wherein said controller controls said shockwave sources such that shockwaves from said shockwave sources reach the common focus at different times.

11. The shockwave device according to claim 1 wherein said shockwave sources operate in series.

12. The shockwave device according to claim 1 wherein said shockwave sources operate in parallel.

13. The shockwave device according to claim 1 wherein said shockwave sources are arranged to lie in a plane generally perpendicular to a longitudinal axis of a patient.

14. The shockwave device according to claim 1 and further comprising imaging apparatus.

15. The shockwave device according to claim 1 and further comprising a patient couch.

16. The shockwave device according to claim 15 wherein said shockwave sources are arranged to lie in a plane generally perpendicular to a longitudinal axis of said couch.

17. The shockwave device according to claim 15 wherein said shockwave sources are arranged with respect to said couch such that a back of a patient lying on said couch is generally perpendicular to a plane in which lie said shockwave propagation axes.

18. The shockwave device according to claim 1 wherein said shockwave sources comprise at least one of an electro-hydraulic shockwave source, an electro-magnetic shockwave source and a piezo-electric shockwave source.

19. The shockwave device according to claim 1 wherein said shockwave sources are pivotally mounted on a shockwave assembly.

20. The shockwave device according to claim 19 and further comprising at least one actuator adapted to rotate at least one of said shockwave sources about a pivot axis in said shockwave assembly.
